(19) United States
(12) Patent Application Publication
(10) Pub. No.: US 2025/0228457 A1
(43) Pub. Date: Jul. 17, 2025

(54) MONITORING SYSTEM AND METHOD FOR REMOTE MONITORING OF PHYSIOLOGICAL HEALTH

(71) Applicant: MONOVO, LLC, Orem, UT (US)

(72) Inventors: STEVEN TAYLOR, Orem, UT (US); ROBERT JOHNSTUN, Orem, UT (US); JONATHAN MUÑOZ, Orem, UT (US); JAROM SHURTLIFF, Orem, UT (US)

(21) Appl. No.: 18/705,439

(22) PCT Filed: Oct. 27, 2022
(86) PCT No.: PCT/US2022/047997

§ 371 Date: Apr. 26, 2024

Related U.S. Application Data
(60) Provisional application No. 63/272,413, filed on Oct. 27, 2021.
(60) Provisional application No. 63/272,428, filed on Oct. 27, 2021.
(60) Provisional application No. 63/272,529, filed on Oct. 27, 2021.
(60) Provisional application No. 63/272,545, filed on Oct. 27, 2021.
(60) Provisional application No. 63/272,552, filed on Oct. 27, 2021.
(60) Provisional application No. 63/272,553, filed on Oct. 27, 2021.
(60) Provisional application No. 63/272,431, filed on Oct. 27, 2021.

(51) Int. Cl. A61B 5/00

(52) U.S. Cl. CPC A61B 5/0022; A61B 5/6801; A61B 5/7221; A61B 5/7264

(57) ABSTRACT

A health monitoring system and corresponding method for monitoring a physiological condition of a user may include collecting, by a monitoring device, measurements representing physiological signals of the body, transmitting the measurements from the monitoring device to a local device, and receiving, at a remote server, the measurements and additional data from the local device. The remote server may include a processing module, a review portal and a clinician portal, such that the remote server may be arranged to automatically classify the measurements representing physiological signals of the body, verify the classifications, and provide the verified measurements at a clinician portal.

### CROSS-REFERENCE TO RELATED APPLICATION

This application claims priority to and the benefit of United States Provisional Patent Application Nos. 63/272,529, filed Oct. 27, 2021; 63/272,413, filed Oct. 27, 2021; 63/272,545, filed Oct. 27, 2021; 63/272,552, filed Oct. 27, 2021; 63/272,553, filed Oct. 27, 2021; and 63/272,428, filed Oct. 27, 2021. Each of the foregoing applications is incorporated herein by reference in its entirety.

### BACKGROUND

Remote monitoring of an individual's health is becoming a useful tool in modern healthcare and in personal efforts to improve well-being. Wearable sensors have grown increasingly prevalent and are now commonly used to monitor physiological parameters ranging from a number of steps taken and heart rate to movement during sleep. These wearable sensors can give a user or a healthcare professional improved information regarding an individual's health and do so using measurements taken outside of a clinical setting, from the “real-life” of a subject. These wearable technologies have the potential to improve quality of life, facilitate early intervention for adverse medical conditions, and advance health and medical research.

A recognized impediment to the development and use of these wearable sensors is the effective collection, storage and use of the resulting data. While constant monitoring and collection of physiological signals from a body would appear to offer the most advantageous “monitoring” of a user's health, in reality the sheer quantity of data produced in such a scenario significantly impedes effective monitoring by masking concerning signals and/or requiring complex and expensive devices for data collection and storage. This challenge is further magnified by security and privacy concerns, as recorded health data is sensitive information protected by law and regulation, such that the storage and transfer of health data cannot be handled in a conventional or routine manner.

Existing monitoring systems are limited by these challenges, such that data provided by wearable devices provides only limited benefits. In some instances, wearable devices have been provided including a memory for storing collected measurements. These wearable devices may be removed and sent to a health service provider for evaluation, for example every two weeks. This approach has the disadvantage that the “monitoring” provided is intermittent at best, such that benefits of the method are limited over a regular visit to a primary care provider.

In other implementations, conventional systems may rely on the calculations of averages, time limited selections of measurements, or on random sampling of measurement data for evaluation. While such methods reduce the amount of data for evaluation, they likewise limit the effectiveness of the monitoring as the measurements present only a limited view of the user's health. Further, existing methods as described above all require significant intervention from a clinician, such that the cost and inefficiencies of these methods is unreasonably high.

In like manner, conventional monitoring systems do not provide an effective way to interface with the user. If the health monitoring according to existing methods identifies an issue with the measurements or with the user's health condition, an individual clinician must identify the issue and initiate communication with the user to gather more information or to inform the user of a problem.

From the foregoing, there is a need for an improved health monitoring system and method for remote monitoring of a user's physiological condition that enables a more real-time monitoring of physiological measurements. There is further a need for a system and method capable of reducing the demands on clinicians, by reducing the amount of information presented to a clinician while still maintaining essentially real-time or continuous monitoring.

### BRIEF SUMMARY

According to embodiments of the disclosure, a health monitoring system may include a monitoring device arranged for measuring physiological signals from a body and generating an output including at least a first measurement. The monitoring device may be a wearable device placed against a user's body in a manner such that the device may collect regular or real-time physiological measurements from the body, for example using a sensor of the monitoring device. The monitoring device may be arranged to communicate with a local device, such as over a first communication network. Together, the monitoring device and the local device provide an improved collection of physiological signals from a body, as the functions of each device may be configured to be complementary and/or collaborative. Advantageously, this arrangement increases the operational abilities of each of the monitoring device and the local device, even if conventional devices are employed in the system.

The local device of the health monitoring system may provide the output from the monitoring device to a remote server, either as received from the monitoring device or as a modified output, such as a personalized output. The local device may generate a personalized output on the basis of the output received from the monitoring device and one of a personal identifier, a device identifier or the like. The local device and the remote server may be in communication over a second communication network.

In embodiments of the health monitoring system, the remote server may be configured to receive and process the output or personalized output received from the local device. The remote server may include a processing module, a review portal, and a clinician portal. The processing module may be arranged to automatically evaluate and classify the first measurement, for example as one of an artifact, a potential problem or as having no apparent evidence of abnormality (NAEOA). A tag or other identifier may be assigned to the first measurement on the basis of the classification, such that the first measurement may be stored with or otherwise associated with said classification.

A personalized output received from the local device may be processed at the processing module of the remote server on the basis of the first measurement and one or more additional characteristics of the user. These characteristics may include further information collected by the local device regarding the output of the monitoring device, information from the user's medical history, preferences identified by the user or a clinician, and/or privacy requirements related to the user.

The review portal may be configured to receive the first measurement and the associated classification from the processing module, and to perform a verification of the classification. The review portal may present the first measurement and the corresponding tag for verification, such as by a review technician, clinician or a review module of the remote server. The review portal may automatically verify a classification of the first measurement as an artifact or as having no apparent evidence of abnormality without review or input to the review portal. Alternatively, the review portal may only verify the classification of the first measurement where the first measurement is classified as a potential problem.

The clinician portal of the remote server may be configured to present the verified first measurement and the corresponding tag, such as to inform a clinician. The verified first measurement may be presented to the clinician in a manner corresponding to the tag associated with the measurement. For example, a first measurement classified and verified as having no apparent evidence of abnormality may be presented to the clinician by storage in a memory accessible to the clinician through the clinician portal, while a first measurement classified and verified as a potential problem may be presented by the clinician portal through a direct communication to the clinician.

Embodiments of the monitoring device, the local device and the remote server may each include one or more processors, one or more computer readable media, one or more communication interface, and/or similar components. The monitoring device, the local device and the remote server may be provided with programmable instructions stored in the computer readable media, such that the one or more processors may enable the above-described operations on the basis of said instructions.

Embodiments of a health monitoring system may be used to perform a corresponding method for monitoring physiological signals of a user's body according to the present disclosure. Methods for monitoring physiological signals of a user's body may include recording, processing and/or transmitting physiological signals collected from a user's body as a first measurement. The first measurement may be automatically classified as one of an artifact, a potential problem or a normal measurement and assigned a corresponding tag.

The method may include verifying the classification of the first measurement at a reviewer portal accessible to a review technician and presenting the measurements and verified classifications at a clinician portal.

The above embodiments solve the problem of health monitoring systems that are difficult to use, by providing an improved health monitoring system and method for monitoring a physiological condition of a user with increased efficiency and simplicity. The embodiments allow an advantageous filtering, or automatic processing, of regularly collected or real-time measurements collected by a monitoring device while reducing input required from a clinician and an amount of information provided to the clinician. The health monitoring system and method further improves the efficacy of health monitoring by enabling efficient, real-time monitoring by a clinician-controlled system using a unique arrangement of devices, which at least in some possible embodiments may include primarily conventional components.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not, therefore, to be considered to be limiting in scope, embodiments will be described and explained with additional specificity and details through the use of the accompanying drawings in which:

FIG. 1 is a simplified diagrammatic view of a health monitoring system according to embodiments of the disclosure.

FIG. 2 is a simplified diagrammatic view of a monitoring device according to the embodiment of FIG. 1.

FIG. 3 is a simplified diagrammatic view of a local device according to the embodiment of FIG. 1.

FIG. 4 is a flow diagram of operations performed by a remote server according to the embodiment of FIG. 1.

FIG. 5 is a flow diagram of operations performed by a remote server according to a further embodiment of the disclosure.

FIG. 6 is a flow diagram of a method for monitoring a physiological condition of a user according to embodiments of the disclosure.

The drawings and figures are not drawn to scale, but instead are drawn to provide a better understanding of the components, and are not intended to be limiting in scope, but to provide exemplary illustrations. The figures illustrate exemplary configurations of a health monitoring system and associated method, and in no way limit the structures or configurations of a body interface and components according to the present disclosure.

### DETAILED DESCRIPTION

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. The connections and arrangements represented in the figures introduced above are to be understood as exemplary and are not necessarily shown in proportion or in exhaustive detail. It should be understood, however, there is no intention to limit the disclosure to the specific embodiments disclosed, but on the contrary, the intention covers all modifications, alternative constructions, combinations, and equivalents falling within the spirit and scope of the disclosure.

The flowchart illustrations and block diagrams in the flow diagrams illustrate the architecture, functionality, and operation of possible implementations of systems, methods, and computer program products according to various embodiments of the present disclosure. In this regard, each block in the flowchart illustrations or block diagrams may represent a module, segment, or portion of code, which comprises one or more executable instructions for implementing the specified logical function(s).

It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable media that can direct a computer or other programmable data processing apparatus to function in a particular manner, such that the instructions stored in the computer-readable media produce an article of manufacture including instruction means which implement the function/act specified in the flowchart illustrations and/or block diagram block or blocks.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the only relevant components of the device are A and B.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment but may refer to more than one embodiment. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the embodiments require more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some, but not other features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description. Examples of the current disclosure may be provided in terms referring to an electrocardiogram (ECG) measurement, such as including a classification of a measurement as an artifact, a potential problem or as having no apparent evidence of an abnormality, however there is no intention to limit the disclosure thereto. Rather, one of ordinary skill in the art will recognize the possibility of applying the same principles of embodiments of the current disclosure to alternative and additional physiological parameters and classifications.

Embodiments of a health monitoring system and related methods are provided for monitoring a physiological condition of a user with an increased level of efficiency, while reducing input required from a clinician or other healthcare provider.

The embodiments of the health monitoring system and the related methods according to the present disclosure advantageously facilitate regular or real-time monitoring by automatically processing, classifying and verifying measurements recorded from a user's body. Automatically processing, classifying and verifying measurements advantageously provides an effective filtering of real-time data into actionable information for presentation to a clinician. Thus, monitoring of relevant measurements by a clinician is increased, while the required input from a clinician is reduced.

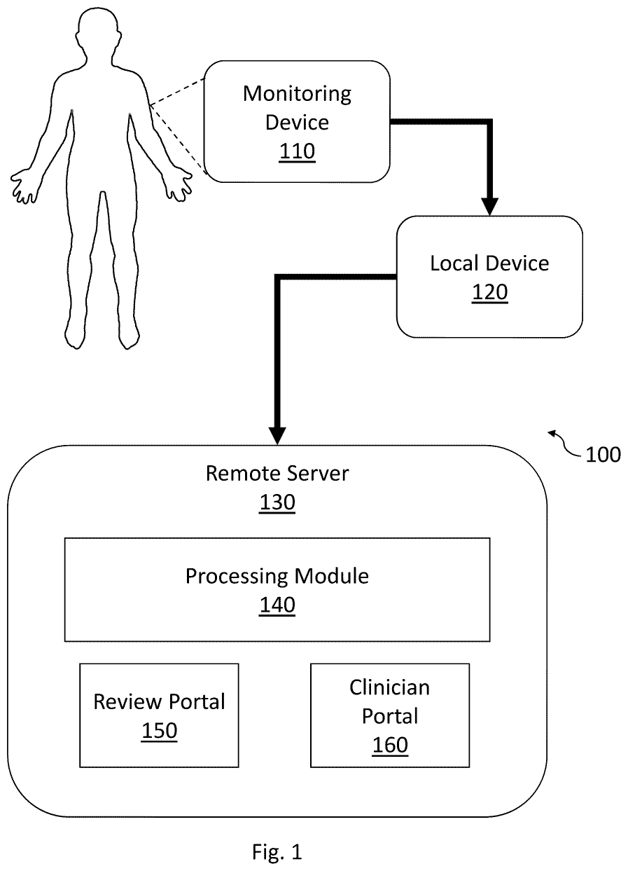

FIG. 1 illustrates an embodiment of a health monitoring system 100 including a monitoring device 110 in communication with a local device 120 for collecting and recording physiological signals from a user's body as at least a first measurement. The local device 120 may be configured to provide the output from the monitoring device 110 to a remote server 130, either as received from the monitoring device 110 or as a modified output, such as a personalized output including an identifier unique to the monitoring device, the local device, and/or the user. The monitoring device 110 and the local device 120 may be operatively connected through a first communication network, and the local device 120 and the remote server 130 may be operatively connected through a second communication network.

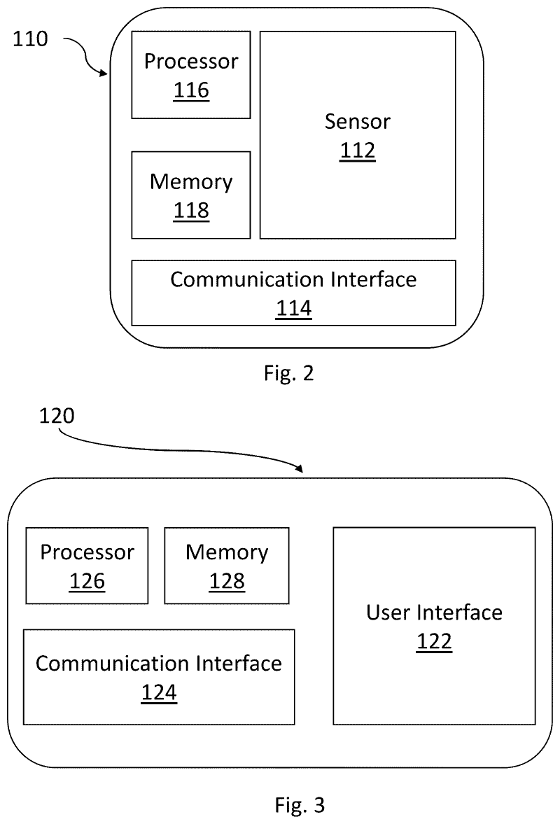

As illustrated in FIG. 2, a monitoring device 110 may generally include a sensor 112 and a communication interface 114 for communicating through the first communication network. According to varying embodiments, the monitoring device 110 may further include one or more processors 116 and/or one or more computer readable media 118, such as a memory. The monitoring device 110 may be provided in the form of a wearable device, such as may be worn on the wrist or the chest, and may be provided with a power source or battery (not shown) to facilitate independent operation.

In operation, the monitoring device 110 may be arranged to collect measurements from physiological signals of a body using the sensor 112, such that the collected measurements may be stored in the computer readable media 118 and/or communicated to the local device 120 using the communication interface 114. The sensor 112 of the monitoring device 110 may include any suitable sensor that may be arranged for monitoring physiological signals from the body of the user or the like, as would be recognized by one skilled in the art. Such sensors may include light sensors, sound sensors, pressure sensors, humidity sensors, position sensors, piezo film sensors, force sensors, temperature sensors, motion sensors, etc.

Similarly, the monitoring device 110 may include a plurality of sensors 112, such as for collecting measurements corresponding to different physiological signals or condition. In a further embodiment, the monitoring device 110 may include a plurality of sensors 112 as redundant sensors 112, such that the plurality of sensors 112 may facilitate multiple measurements for representing the same physiological signal or condition. The use of multiple sensors 112, and particularly redundant sensors 112, advantageously enables automatic verification of the measurements collected by the monitoring device 110 and improves both the speed and accuracy of reported measurements relative to conventional systems. Redundant measurements may be combined or otherwise processed together at the monitoring device 110 and/or the local device 120 and/or the remote server 130, as would be understood from the instant disclosure.

In one aspect of the disclosure, the communication interface 114 of the monitoring device 110 may facilitate only a local connection, or may be provided as local communication interface, such that the use of an internet connection, internet service provider (ISP) or other third-party data connection is not required for communications between the monitoring device 110 and the local device 120. In one embodiment, the communication interface 114 may be restricted to short distance wireless communications.

An embodiment of a local device 120 is shown in FIG. 3, including a one or more processors 126, one or more computer readable media 128, such as a memory, and a communication interface 124 for communicating through the first communication network. According to varying embodiments, the local device 120 may further include a user interface 122 including button inputs, a touch screen, camera or other reading device, speakers, microphone or other components as would be understood by one of ordinary skill in the art. In one example, the local device 120 may be a mobile computing device, such as a mobile phone or a laptop computer.

In operation, the local device 120 may be arranged to receive an output from the monitoring device 110 through the first communication network using the communication interface 124, the output including at least a first measurement collected by the monitoring device 110 from the body. The local device 120 may store the output from the monitoring device 110 in the computer readable media 128, generate a modified or personalized output on the basis of the output from the monitoring device 110, and/or communicate the output or the personalized output to the remote server 130 using the communication interface 124. According to embodiments, the communication interface 124 of the local device 120 may include a plurality of communication interfaces or a single communication interface configured for communicating with both the monitoring device 110 over the first communication network and the remote server 130 over the second communication network. The communication interface 124 of the local device 120 may include any interface suitable for electronic communication, such as over a short-range wireless network, a long-range wireless network (i.e., mobile data network, internet network, etc.) or a wired connection, such that the local device 120 is able to transmit and receive information between the monitoring device 110 and the remote server 130. One or more of the monitoring device 110, the local device 120 and the remote server 130 may further be arranged to communicate using a restricted or secure connection, such as peer-to-peer communication, an encrypted communication, etc.

According to alternative embodiments, it is appreciated that the monitoring device 110 and the local device 120 may be implemented as effectively comprising a single computing system, such that the monitoring device 110 is integrated into a single device with the local device 120. In integrated embodiments, the monitoring device 110 and the local device 120 may be operated using the same processor and/or the same memory, such as in an embodiment where a monitoring device 110 forms a peripheral sensor of the local device 120.

As illustrated in FIG. 1, in embodiments of the health monitoring system 100, the remote server 130 may be configured to receive and process the output or personalized output received from the local device 120. The remote server may include a processing module 140, a review portal 150, and a clinician portal 160. Although not illustrated for the sake of simplicity, the remote server 130 may include one or more processors, one or more computer readable media, one or more communication interfaces, and/or similar components. The remote server 130 may further include programmable instructions stored in the computer readable media, such that the one or more processors may facilitate operation of the processing module 140, the review portal 150, and the clinician portal 160.

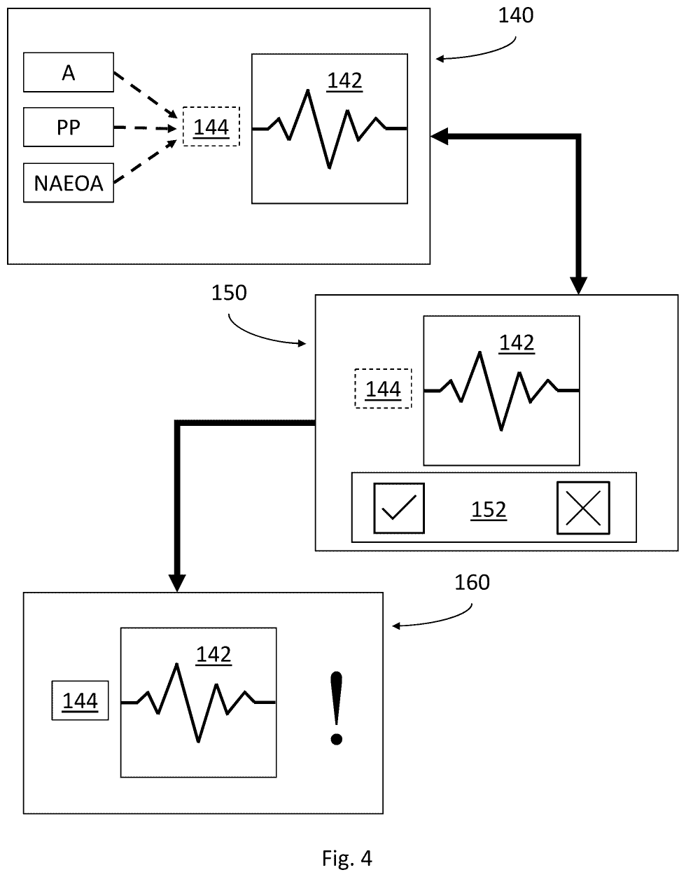

As illustrated in FIG. 4, the processing module 140 may be arranged to automatically evaluate and classify at least the measurement 142 included in the output or personalized output, for example as one of an artifact (A), a potential problem (PP) or as having no apparent evidence of abnormality (NAEOA). A tag 144 or other identifier may be assigned to the measurement 142 on the basis of the classification, such that the measurement 142 may be stored with or otherwise associated with said classification. The processing module 140 may further output the measurement 142 and tag 144 as a readable image or document.

In varying embodiments, the processing module 140 may be configured to recognize from the output or personalized output received from the local device 120 what kind of measurement is provided, characteristics of the body being monitored, and a mapping between properties of the measurement being evaluated and properties of reference measurements classified as an artifact, a potential problem and/or as having no apparent evidence of abnormality. The information can then be used to automatically classify the measurement provided as an artifact, a potential problem and/or as having no apparent evidence of abnormality. This advantageously reduces the cost, complexity, and time-intensity of manually classifying measurements as is required in conventional systems.

Embodiments of the processing module 140 may be arranged to receive and/or automatically classify measurements with respect to a priority level assigned to the measurement. The priority level may be assigned at any of the monitoring device 110, the local device 120, or the remote server 130, such that certain measurements may be evaluated or processed in an expedited manner relative to other measurements. Alternatively, measurements assigned a higher priority level may be provided directly to the clinician portal without verification at the review portal. A priority level may be assigned to a given measurement based on predetermined criteria, such as identifying a measurement that corresponds to an emergency medical condition as a high priority.

The review portal 150 may be configured to receive the classified measurement 142 and the associated tag 144 from the processing module 140, and to perform a verification of the classification. The review portal 150 may present the measurement and the corresponding tag 144 for verification, such as by a review technician, clinician or a review module of the remote server 130. For example, the review portal 150 may present a review interface 152 for receiving an input verifying or changing the classification of the measurement, such as using a touch screen, keyboard or other input device.

In an embodiment, the review portal 150 may automatically verify a classification of the first measurement as an artifact or as having no apparent evidence of abnormality without review or input to the review portal. Alternatively, the review portal 150 may only verify the classification of the measurement where the measurement is classified as a potential problem.

In a further aspect of the current disclosure, an input made at the review interface of the review portal 150 may be provided to the processing module 140 as a reference measurement, such that the accuracy of the classification by the processing module 140 may increase with use, further reducing the intervention required from a reviewer or clinician.

The clinician portal 160 of the remote server 130 may be configured to present the verified measurement 142 and the corresponding tag 144, such as to inform a clinician. The verified measurement 142 may be presented to the clinician in a manner corresponding to the tag 144 associated with the measurement. For example, a measurement classified and verified as having no apparent evidence of abnormality may be presented to the clinician by storage in a memory accessible to the clinician through the clinician portal 160, while a measurement classified and verified as a potential problem may be presented by the clinician portal 160 through a direct communication to the clinician.

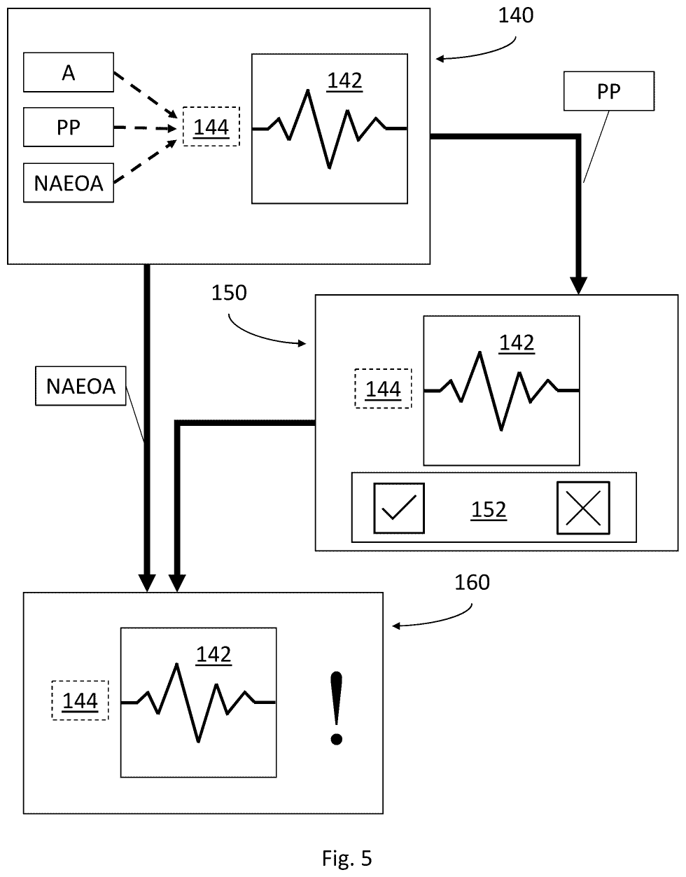

The embodiment of FIG. 5 differs from FIG. 4 in that the review portal 150 is employed to verify the classification of measurements according to a predetermined filter. For example, as depicted, measurements tagged as potential problems are provided at the review portal 150 for review while measurements tagged as having no apparent evidence of abnormality are not. In like manner, the remote server 130 may be configured to provide only verified measurements with predetermined tags for clinician review at the clinician portal 160.

The disclosure of the present application may be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

As such, the described remote server 130 may be provided as a cloud computing environment and may include the described processing module 140, review portal 150, and clinician portal 160 as virtual machines such as may be accessible to a reviewer and/or clinician by means of a suitable computing device, for example desktop computer, laptop computer or mobile device as would be understood from the current disclosure by one skilled in the art. For example, a clinician may access the clinician portal 160 using an application executed on a mobile device or smartphone.

By providing an automated system for classifying and verifying measurements from a monitoring device according to disclosed embodiments, the problem of existing approaches to remote health monitoring, including costly, complex, and time-intensive manual methods for reviewing measurements from a monitoring device, are addressed. The disclosed embodiments advantageously provide an automated system configured to receive an output including a measurement from a monitoring device, classify the measurement, verify the classification, and provide a clinician with access to verified measurements, with significantly reduced cost and complexity compared to existing approaches.

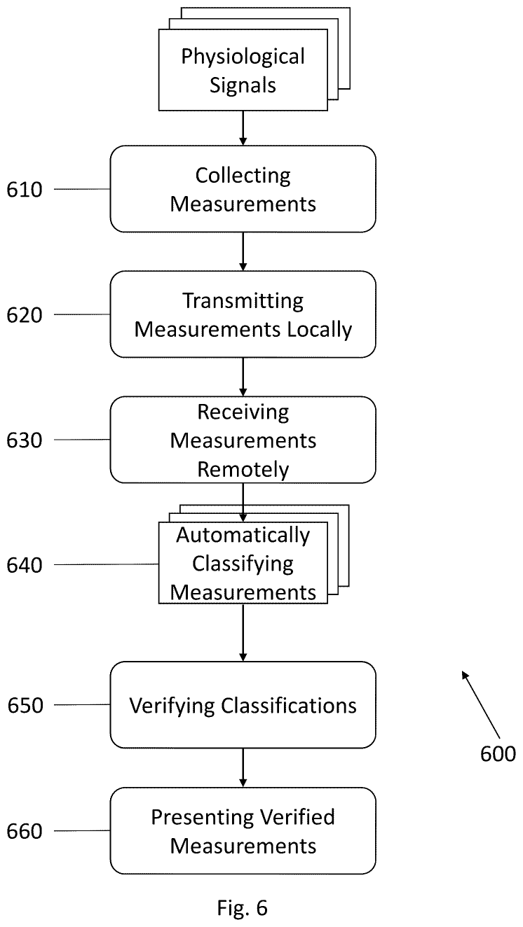

FIG. 6 illustrates a method 600 that may be performed by a health monitoring system such as described above, for remote monitoring of a physiological condition of a user. The method 600 may include collecting 610, such as by a monitoring device, measurements representing physiological signals of a body. Physiological signals may include various indicators of physiological conditions, such as heart rate, respiration, blood oxygen level, movement state, location, temperature, blood pressure, etc., as would be understood by one skilled in the art from the present disclosure. The transmitting step 620 may involve a substantially continuous communication between the monitoring device and the local device or may occur at a predetermined interval of time or based on an amount of measurement data recorded.

The measurements representing physiological signals of the body may then be transmitted 620 to a local device. As discussed previously, it is noted that embodiments are contemplated where the local device and the monitoring device are provided as a single device, such that the transmitting step 620 may or may not be required. However, in various embodiments it is preferred to provide the local device and the monitoring device as distinct physical devices, such as in the form of a wearable device and a mobile phone, necessitating the transmitting step 620.

The step of collecting 610 the measurements may include recording, compiling, processing and/or storing the measurements, whether at the monitoring device, the local device or some combination of the two. As such, the measurements may comprise an averaged value over a predetermined period of time, may include a set of measurements covering a predetermined period of time, may be arranged to include a timestamp, or otherwise modified for communication and evaluation as preferred for the requirements of a given physiological parameter. Similarly, additional information may be added to the measurements for assisting in processing of the measurements. For example, a device or user identifier may be included in the measurements, the identifier being associated with a user or the device and information from the user's medical history, preferences or concerns identified by the user or a clinician, and/or privacy requirements related to the user. In this way a more accurate evaluation of the measurements may be performed, and further information may be provided informing a clinician, the user or the health monitoring system of appropriate next steps.

The method 600 may include receiving 630, such as at a remote server or cloud system, the measurements and further information as provided by the local device and/or the monitoring device. The measurements may then be automatically classified 640 as one of an artifact, a potential problem, a normal measurement, or similar classification, and assigned a corresponding tag, such as at a processing module of the remote server. The automatic classification 640 may be based on a comparison of the measurements with a plurality of reference measurements or standards. The automatic classification 640 may further be adjusted based on additional information, such as a comparison of multiple measurements from the monitoring device, information from the user's medical history, and/or preferences or concerns identified by the user or a clinician. For example, a clinician may have set a base level or an alarm level for an individual user, or a measurement may be treated differently based on whether the user is shown to be walking, sitting, or sleeping when the measurement was recorded. The method may therefore be advantageously configured to specific medical concerns for a given user or adapted to apply defined population-based information in the classification step, offering a previously unattainable flexibility, accuracy and efficiency in an automatic classifying step.

According to embodiments, the classification of the measurements may be verified 650. The verification may be performed only for certain classifications, such as only for potential problems, or may differ in the level of verification required based on the classification. For example, only potential problems may be directly provided to a review technician for verification through a review portal, while other classifications may be automatically verified or may be stored for non-priority verification at the convenience of a clinician or reviewer.

A step of presenting the measurements and verified classifications 660, such as at a clinician portal accessible to a clinician, may be provided in the method 600. The verified measurements and the corresponding tags may be provided in a predetermined manner, such as to inform a clinician of a particular level of concern. For example, the verified measurement may be presented to the clinician in a manner corresponding to the tag associated with the measurement. Accordingly, a measurement classified and verified as having no apparent evidence of abnormality may be presented to the clinician by storage in a memory accessible to the clinician through the clinician portal, while a measurement classified and verified as a potential problem may be presented by the clinician portal through a direct communication to the clinician, e.g. by sending a direct communication to the clinician through a call, email, text, a notification at a corresponding application of a mobile device, etc.

The system and method of the current application advantageously reduce the time-intensity of managing and monitoring measurements from a monitoring device, particularly in regular or real-time monitoring. The system and method embodiments according to the disclosure advantageously improve the functionality of a computer system on or in cooperation with which the automated system is carried out by providing specific rules allowing for automatic evaluation and action in response to the physiological condition of a user, reducing the processing requirements of the system while improving the efficiency of remote monitoring.

The skilled artisan will recognize the interchangeability of various components and steps from different embodiments described. Besides the variations described, other known equivalents for each feature can be mixed and matched by one of ordinary skill in this art to construct or use an automated system and method under principles of the present disclosure. Therefore, the embodiments described may be adapted to regulatory and standard requirements in general, including requirements across medical devices, pharmaceuticals, diagnostics, physical therapies, medical interventions, and other fields.

1. A method for monitoring a physiological condition from physiological signals collected from a body, the method comprising:
optionally, collecting, by a monitoring device, measurements representing said physiological signals of the body;
optionally, transmitting the measurements from the monitoring device to a local device;
receiving, at a remote server, the measurements and a local device identifier from the local device;
automatically classifying each of the measurements as one of an artifact, a potential problem or a normal measurement and assigning a corresponding tag to the respective measurement at a processing module of the remote server;
verifying the classification of the measurements at a reviewer portal of the remote server accessible to a review technician; and
presenting the measurements and verified classifications at a clinician portal accessible to a clinician.

2. The method of claim 1, wherein the monitoring device is a wearable device.

3. The method of claim 1, wherein the monitoring device comprises at least one sensor configured for collecting the measurements representing said physiological signals of the body.

4. The method of claim 1, wherein the monitoring device and the local device are operatively connected by a first communication network.

5. The method of claim 1, wherein the local device and the remote server are operatively connected by a second communication network.

6. The method of claim 1, wherein the local device is configured to add a timestamp to the measurements transmitted by the monitoring device.

7. The method of claim 1, wherein the local device is arranged to provide the measurements to the remote server at a predetermined interval.

8. The method of claim 7, wherein the predetermined interval is less than 24 hours.

9. The method of claim 1, wherein the monitoring device comprises at least two redundant sensors for collecting the measurements.

10. The method of claim 1, wherein the step of verifying the classification of the measurements at the reviewer portal of the remote server comprises verifying at least the potential problem classification of the measurements.

11. The method of claim 1, wherein the step of verifying the classification of the measurements at the reviewer portal of the remote server comprises changing classification of at least one of the measurements.

12. The method of claim 1, wherein the measurements collected by the monitoring device comprise electrocardiogram (ECG) measurements.

13. The method of claim 7, wherein the predetermined interval is between 6 and 12 hours.

14. The method of claim 7, wherein the predetermined interval is between 3 and 5 hours.

15. The method of claim 7, wherein the predetermined interval is between 1 and 2 hours.

16. The method of claim 1, wherein the local device is arranged to continuously provide the measurements to the remote server.

17. The method of claim 1, wherein the step of automatically classifying each of the measurements as one of an artifact, a potential problem or a normal measurement and assigning a corresponding tag to the respective measurement at the processing module of the remote server is based on a set of predetermined measurement parameters set at the processing module.

18. The method of claim 17, wherein the set of predetermined parameters for automatically classifying each of the measurements is selected based on the local device identifier.

19. The method of claim 9, wherein the measurements collected by the at least two redundant sensors comprise at least a first measurement and a second measurement, at least the first measurement and the second measurement being provided as overlay data.

20. A health monitoring system, the monitoring system comprising:
a monitoring device arranged for measuring physiological signals from a body and generating an output including at least a first measurement;
a local device arranged for receiving said output and generating a personalized output including at least the first measurement and a local device identifier;
a remote server configured to receive and process said personalized output, said remote server comprising:
a processing module arranged to automatically classify the first measurement as an artifact, a potential problem or as having no apparent evidence of abnormality (NAEOA), the processing module configured to assign a corresponding tag to the first measurement according to said classification;
a review portal arranged to present the first measurement and the corresponding tag for verification; and
a clinician portal arranged to present the verified first measurement and the corresponding tag.

21. (canceled)
